(19) United States
(12) Patent Application Publication
(10) Pub. No.: US 2025/0198979 A1
(43) Pub. Date: Jun. 19, 2025

(54) MONITORING SYSTEM FOR MONITORING A STRENGTH DEVELOPMENT OF SPRAYED CONCRETE

(71) Applicant: NORMET OY, Iisalmi (FI)

(72) Inventor: Samu KUKKONEN, Iisalmi (FI)

(21) Appl. No.: 18/847,938

(22) PCT Filed: Mar. 9, 2023
(86) PCT No.: PCT/FI2023/050131

§ 371 Date: Sep. 17, 2024

(30) Foreign Application Priority Data
Mar. 17, 2022 (FI) 20225238

(51) Int. Cl. G01N 33/38; E21D 11/10; G08C 17/00

(52) U.S. Cl. CPC G01N 33/383; E21D 11/105; G08C 17/00

(57) ABSTRACT

The application relates to a monitoring system for monitoring a strength development (SD) of sprayed concrete. The system comprises a sprayer for spraying the concrete on a target surface and at least one monitoring sensor for sensing at least one strength development quantity (TQ, MQ) from the concrete. The system further comprises a communicator for receiving at least one sensed strength development quantity-related information (TE, MO) wirelessly from the at least one monitoring sensor and a processor for processing the at least one received strength development quantity-related information to represent the development of the strength of the concrete. The sprayer is further configured to spray the at least one monitoring sensor on the target surface so that the at least one monitoring sensor is among the sprayed concrete after the sprayer has been provided with the at least one monitoring sensor.

### TECHNICAL FIELD OF THE INVENTION

The application relates generally to a monitoring system for monitoring a strength development of sprayed concrete.

### BACKGROUND OF THE INVENTION

Concrete is used to reinforce rock structures in mining and tunneling environments, and it is sprayed as a concrete layer on these structures by means of mobile concrete sprayers.

The concrete sprayers have a movable, telescopic spraying boom that supports a concrete hose through which the concrete flows to a nozzle head of the boom. The nozzle head with a nozzle tip is installed at the end of boom and an operator uses a moving mechanism and a telescopic structure of boom in order to drive the nozzle tip to a desired location, close to a target surface, which will be coated by the concrete layer, so that it is possible to direct a concrete spray accurately to this surface.

An early-age strength of the sprayed concrete is often required for an immediate ground support in the modern mining and tunneling. The speed of advance in the tunneling is strongly influenced by a rate of an early-age strength development of the sprayed concrete since it determines in soft ground and weak rock when excavation heading can proceed.

One solution to assess the development state of setting of the sprayed concrete is to measure a resistance to an early age compressive strength development by indirect methods, e.g. a needle penetration or a nail extraction, which can cause a safety risk to the operator entering under the fresh concrete. Another solution to the assessment is an isothermal calorimetry that measures the enthalpy of reaction or heat flux during concrete hydration.

### SUMMARY

An object of the invention is to withdraw the drawbacks of known solutions and to provide a fast and easy monitoring system to monitor (detect) a strength development of a fresh sprayed concrete as well as to increase safety and to save time and cost by optimizing an entire concrete spraying process by utilizing pressure, temperature, and humidity related parameters.

An object of the invention is fulfilled by providing the monitoring system and monitoring method according to the independent claims.

Embodiments of the invention are specified by the monitoring system and monitoring method according to the independent claims.

A first embodiment comprises a monitoring system for monitoring a strength development of sprayed concrete. The system comprises a sprayer for spraying the concrete on a target surface and at least one monitoring sensor for sensing at least one strength development quantity from the concrete. The system further comprises a communicator for receiving at least one sensed strength development quantity-related information wirelessly from the at least one monitoring sensor and a processor for processing the at least one received strength development quantity-related information to represent the development of the strength of the concrete. The sprayer is further configured to spray the at least one monitoring sensor on the target surface so that the at least one monitoring sensor is among the sprayed concrete after the sprayer has been provided with the at least one monitoring sensor.

A second embodiment comprises a monitoring method for monitoring a strength development of sprayed concrete by the monitoring system according to the previous embodiment. The method comprises at least following steps of spraying, by the sprayer, the concrete on the target surface and sensing, by the each monitoring sensor, the at least one strength development quantity from the sprayed concrete. The method further comprises at least following steps of receiving, by the communicator, the at least one sensed strength development quantity-related information wirelessly from the at least one monitoring sensor and processing, by the processor, the at least one received strength development quantity-related information to represent the development of the strength of the concrete. The at least one monitoring sensor is sprayed, by the sprayer, on the target surface after the sprayer has been provided with the at least one monitoring sensor.

### BRIEF DESCRIPTION OF THE FIGURES

The exemplary embodiments of the invention are described with reference to the following figures:

FIG. 1 presents a monitoring system in an underground tunnel environment

FIG. 2 presents an acquisition of strength-development quantity-related information in the monitoring system

FIG. 3 presents operational parts of a reader

### DETAILED DESCRIPTION OF THE FIGURES

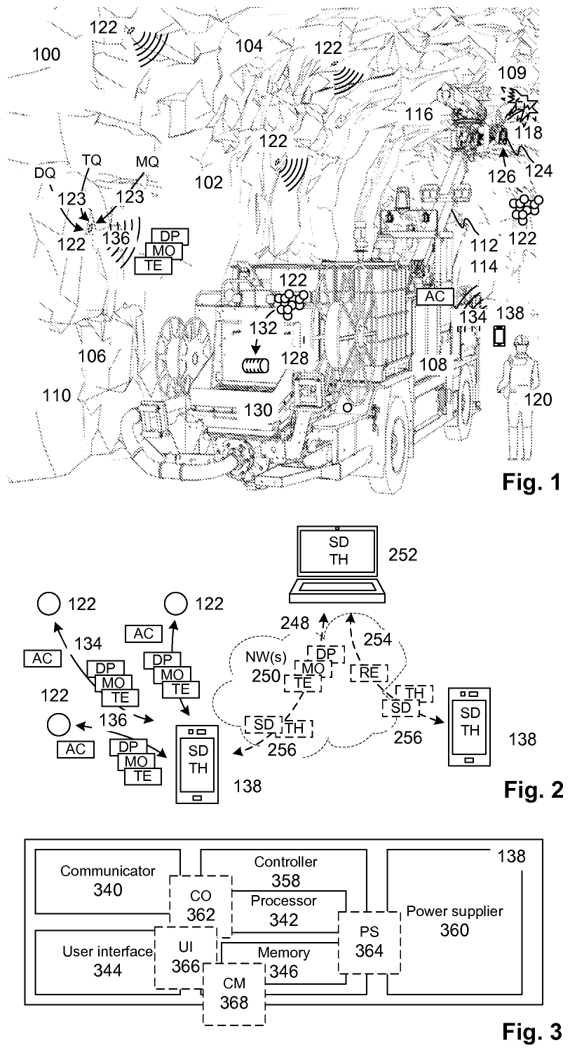

FIG. 1 presents a monitoring system 100 for monitoring a strength development SD of a sprayed concrete 102 on a target surface 104 in a spraying environment 106, which is e.g. a mining environment, a quarry environment, or an underground tunneling environment 106 according to the figure.

The system 100 comprises a sprayer (sprayer machine) 108 that is configured to spray 109 the concrete 102 to form a supporting concrete layer e.g. on the surface 104 of a wall and roof (vault) of the tunnel 110. The sprayer 108 is a mobile concrete sprayer 108 according to the figure that is used in tunnels (tunnel profiles) 110 in the underground mining and tunneling. The sprayer 108 is also configured to spray other materials, e.g. water together with air for cleaning the surface(s) 104 and to operate outside the tunnels 110 in other environments for the same purposes.

The sprayer 108 comprises a telescopic spraying boom 112 that is attached (installed) to the sprayer 108 by means of an attachment mechanism (not presented) to lift and to lower the boom 112 and to rotate (turn around) it by means of its movement mechanism (not presented) in relation to the attachment mechanism.

The boom 112 comprises telescopic boom structures 114 and a nozzle head 116 through which the concrete 102 is sprayed at an end of the telescopic boom structures 114, wherein is a nozzle tip 118.

The boom 112 further comprises several installed location sensors (not presented) that are configured to measure (obtain) location information, which enable an operator 120 of the boom 112 (sprayer 108) to control the boom 112.

The sprayer 108 further comprises a controller (control part, not presented), which is operated by the operator 120, to control movements and operations of the boom 112.

The system 100 further comprises at least one monitoring sensor 122, e.g. one, three, six, nine, or more sensors 122 that is configured to sense (measure) 123 by means of its sensor element(s) at least one strength development quantity, e.g. one, two, three, four, or more quantities, from the concrete 102 and on the grounds of a movement of its place if such exists. The at least one strength development quantity comprises at least one of a temperature TQ of the concrete 102, a moisture MQ of the concrete 102, and a displacement DQ of the monitoring sensor 122 on the surface 104, which, when such exists, reveals whether a thickness of the sprayed concrete 102 is sufficient.

The sprayer 108 is further configured to spray 109 the monitoring sensor(s) 122 on the target surface 104 so that the monitoring sensor(s) 122 is among the sprayed concrete 102 after the sprayer 108 has been provided (supplied) 124, 126 with the monitoring sensor(s) 122. The sprayer 108 may be configured to spray 109 the monitoring sensor(s) 122 among a flow of the concrete 102 or separately without the concrete flow, e.g. in the middle of the temporarily interrupted spraying process.

The system 100 may comprise a sensor injector (cartridge) 124 that is configured to inject the monitoring sensor(s) 122 in the sprayer system either among the concrete flow during the concrete spraying or separately after the injector 124 has been provided 126 by the monitoring sensor(s) 122 to be sprayed. The injector 124 is installed in the nozzle head 116 close the nozzle tip 118.

Alternatively, the system 100 may comprise a sensor injector (cartridge) 128 that is configured to inject the monitoring sensor(s) 122 among the concrete 102 in a concrete hopper 130 of the sprayer 108 after the injector 128 has been provided 132 by the monitoring sensor(s) 122. The injector 128 is installed in the hopper 130 so that the injector and hopper 128, 130 establish a hopper injector 128, 130, wherein the hopper 130 receives the monitoring sensor(s) 122 from the injector 128.

Alternatively, the system 100 may lack the actual injector 124, 128 and the hopper 130 operates as a hopper injector when the operator 120 injects manually the monitoring sensor(s) 122 into the hopper 130 among the concrete 102 or when the monitoring sensor(s) 122 has been injected among the concrete 102 in a concrete batching plant (not presented) so that the concrete 102 and the injected monitoring sensor(s) 122 have been tipped into the hopper 130 later on.

Irrespective of how the monitoring sensor(s) 122 have been provided in the sprayer 108, the operator 120 controls the sprayer 108 to spray 109 the concrete 102 and the monitoring sensor(s) 122 on the surface 104 so that the monitoring sensor(s) 122 are among the sprayed concrete 102.

The power source-comprised monitoring sensor(s) 122 has been activated 134 to operate before the spraying on the surface 104, when the injector 124, 128 or the hopper 130 is provided with the monitoring sensor(s) 122 or when the monitoring sensor(s) 122 are already in the injector 124, 128 or in the hopper 130. Alternatively, the power source-comprised monitoring sensor(s) 122 are activated 134 when those already are among the sprayed concrete 102 on the surface 104. The activation of the monitoring sensor(s) 122 will be described below more accurately.

Each activated monitoring sensor 122 on the surface 104 is configured to start to sense 123 at least one of the strength development quantities TQ, MQ, DQ from the sprayed concrete 102 as previously has been described. Each monitoring sensor 122 is further configured to generate by means of its processor(s) and memory(-ies) a strength development quantity-related information TE, MO, DP, which comprises at least one of temperature-related information TE, moisture-related information MO, or displacement-related information DP, on the grounds of each sensed strength development quantity TQ, MQ, DQ and to transmit (broadcast) 136 by means of its short-range communicator the sensed information TE, MO, DP wirelessly by using a short-range communication, e.g. a radio frequency (RF)-based communication, e.g. Bluetooth, Ultra Wideband (UWB), wireless network protocols (Wi-Fi), or ZigBee communication, or an infrared (IR)-based communication. Additionally, each monitoring sensor 122 is configured receive 134 by means of its short-range communicator an activation message AC wirelessly, if such activation method is used in the system 100.

The system 100 further comprises a reader 138 that is configured to receive 136 by means of its communicator 340 each transmitted information TE, MO, DP wirelessly from the monitoring sensor(s) 122. The reader 138 is further configured to process (use) by means of its processor 342 each received information TE, MO, DP and to use by means of its processor 342 a strength development information, e.g. Early Aged Strength (EAS) information (table), stored in its memory 346 together with said processed information TE, MO to determine the strength development SD of the concrete 102 as well as to use the processed information DP to determine the thickness TH of the concrete layer. Additionally, the reader 138 is further configured to represent by means of its user interface (UI) 344 the determined strength development and thickness information SD, TH of the sprayed concrete 102 to inform a user of the reader 138, e.g. the operator 120 or other operator (person) in the environment 106, the strength and thickness of the sprayed concrete 102 on the surface 104 easily and in real-time.

The reader 138 is further configured to activate 134 by means of its communicator 340 the monitoring sensor(s) 122, which are in the injector 124, 128 before the spraying, in the hopper 130 before the spraying, or on the surface 104 among the sprayed concrete 102 according to the figure, by transmitting (broadcasting) 134 the activation message AC to the monitoring sensor(s) 122 wirelessly by using the short-range communication, e.g. the RF-based communication, e.g. the Bluetooth, UWB, Wi-Fi, or ZigBee communication, or the IR-based communication.

Alternatively or additionally, the injector 124, 128 is configured to activate by means of its short-range communicator the monitoring sensor(s) 122, which are in the injector 124, 128 before the spraying, wirelessly by using the corresponding short-range communication as the reader 138 previously.

Alternatively or additionally to the previous, the operator 120 may activate the monitoring sensor(s) 122 manually by means of its user interface, e.g. an activation button or switch, or by means of contact-free activation, e.g. a magnetic or radio waves activation, before arranging the monitoring sensor(s) 122 in the injector 124, 128 and among the concrete flow or before adding the monitoring sensor(s) 122 among the concrete 102 stored in the hopper 130.

FIG. 2 presents the previously described method how the reader 138 may acquire the determined strength development information SD and thickness information TH of the sprayed concrete 102 directly from the monitoring sensor(s) 122.

Alternatively or additionally to the previous acquisition method, the reader 138 is configured to forward 248 by means of its communicator 340 each information TE, MO, DP, which it has received from the monitoring sensor(s) 122, wirelessly through at least one communication network 250, e.g. one, two, three, four, or more networks 250, to a remote analysis (monitoring) computer 252.

The computer 252 is configured to receive 248 the information TE, MO, DP from several readers 138 that locate same or different sections of the environment 106, or even different environments 106, and it is configured to process by means of its processor each received information TE, MO, DP and to use by means of its processor the strength development information, e.g. the EAS information table, stored in its memory together with said processed information TE, MO to determine the strength development SD of the concrete 102 as well as to use by means of its processor the processed information DP to determine the thickness TH of the concrete mixture in the environment(s) 106. Additionally, the computer 252 is further configured to represent by means of its user interface the determined strength development and thickness information SD, TH of the sprayed concrete 102 to inform a remote user (operator 120) the strength and thickness of the sprayed concrete 102 on the surface(s) 104 in the relevant environment(s) 106 easily and in real-time.

Additionally, if the reader 138, which has forwarded 248 the information TE, MO, DP and is unable to determine independently the strength development SD and thickness TH of the concrete 102 for some reason, or other reader 138, which has not even received any information TE, MO, DP from the monitoring sensor(s) 122, or any other communication device transmits 254 by means of its communicator 340 a strength development and thickness information request RE, the computer 252 is further configured to transmit 256 by means of its communicator on request the strength development and thickness information SD, TH of the concrete 102 in the relevant environment 106 on the grounds of the location of the requesting reader 138 or device.

FIG. 3 presents operational parts of the wireless, portable (mobile) reader 138, which is e.g. wireless smartphone, tablet computer, or laptop computer.

The reader 138 comprises a controller 358 that is configured to control the operations of e.g. reader parts 340, 342, 344, 346, 360 so that the reader 138 operates as previously and below has been described.

The controller 358 comprises the previously described processor 342 that is configured to carry out operator-initiated instructions, computer program (application, software)-initiated instructions, or both, and to process data to run computer programs. The processor 358 comprises at least one processor, e.g. one, two, three, four, or more processors.

The controller 358 further comprises the previously-described memory 346 that is configured to store and to maintain data. The data may be instructions, computer programs, and data files. The memory 346 comprises at least one memory, e.g. one, two, three, four, or more memories.

The reader 138 further comprises the previously-described communicator 340 that the controller 358 is configured to control to send commands, requests, and data to at least one of e.g. the monitoring sensor(s) 122 and the computer 252 in the system 100. The controller 358 is further configured to control the communicator 340 to receive commands, requests, and data from at least one of e.g. the monitoring sensor(s) 122 and the computer 252. The communication between the communicator 340 and at least one of the monitoring sensor(s) 122 and the computer 252 in the system 100 is carried out through a wired connection(s), wireless connection(s), or both connections.

The reader 138 further comprises a power supplier 360 that the controller 358 is configured to control to power the operation of the reader 138. The power supplier 360 comprises at least one supplier part to power the reader 138, e.g. a connection to electric plugs, battery, regulator, or other supplier part.

The reader 138 further comprises the previously-described UI 344 that the controller 358 is configured to control to receive instructions, requests, or data from e.g. the operator 120 or other person(s). The controller 358 is further configured to control the UI 344 to present instructions, requests, or data to e.g. the operator 120 or other person(s).

The memory 346 is configured to store at least a communicator program 362 to operate (control) the communicator 340, a power supplier program 364 to operate the power supplier 360, and an UI program 366 to operate the UI 344.

The memory 350 is further configured to store a computer program 368 that the controller 358 is configured to execute (run, carry out) to control the operation of the reader 138 as previously has been described. The computer program 368 comprises computer readable code instructions.

The computer program 368 may be stored in a tangible, non-volatile (nontransitory) computer-readable medium, e.g. a Compact disc (CD) or Universal Serial Bus (USB) storage device.

The invention and its several advantages have now been described with reference to the previous exemplary embodiments. It is clear that the invention is not only restricted to these embodiments but it comprises all possible embodiments within the scope of the following claims.

1. A monitoring system for monitoring a strength development of sprayed concrete, comprising:
a sprayer for spraying the concrete on a target surface,
at least one monitoring sensor for sensing at least one strength development quantity from the concrete,
a communicator for receiving at least one sensed strength development quantity-related information wirelessly from the at least one monitoring sensor, and
a processor for processing the at least one received strength development quantity-related information to represent the development of the strength of the concrete,
wherein the sprayer is further configured to spray the at least one monitoring sensor on the target surface so that the at least one monitoring sensor is among the sprayed concrete after the sprayer has been provided with the at least one monitoring sensor.

2. The monitoring system according to claim 1, wherein the sprayer is further configured to spray the at least one monitoring sensor among a flow of the concrete.

3. The monitoring system according to claim 1, which further comprises a sensor injector that is configured to inject the at least one monitoring sensor among the concrete to be sprayed.

4. The monitoring system according claim 3, wherein the sensor injector comprises a concrete hopper that is configured to receive the at least one monitoring sensor from a hopper injector or manually from an operator to inject the at least one received monitoring sensor among the concrete in the concrete hopper.

5. The monitoring system according claim 3, wherein the sensor injector comprises a sprayer injector that is attached to the sprayer and configured to inject the at least one monitoring sensor among a flow of concrete during the spraying.

6. The monitoring system according to claim 1, wherein the at least one monitoring sensor is further configured to be activated to sense the at least one strength development quantity from the concrete by a reader, by a hopper injector, or manually by an operator before the spraying, when the at least one monitoring sensor is added among the concrete stored in a concrete hopper, or by a sprayer injector, when the at least one monitoring sensor is added among a flow of concrete during the spraying.

7. The monitoring system according to claim 1, wherein the communicator is further configured to activate the at least one detection sensor to sense the at least one strength development quantity from the concrete.

8. The monitoring system according to claim 1, wherein each monitoring sensor is further configured to transmit the strength development quantity-related information wirelessly.

9. The monitoring system according to claim 1, wherein each monitoring sensor is further configured to generate the strength development quantity-related information on the grounds of the at least one strength development quantity, which comprises at least one of a temperature and a moisture, and the processor is further configured to use the strength development quantity-related information received from the at least one monitoring sensor together with a strength development information to determine the strength development of the concrete.

10. The monitoring system according to claim 1, wherein the processor is further configured to represent the determined strength development of the concrete by a display to inform the strength of the sprayed concrete on the target surface.

11. The monitoring system according to claim 1, wherein each monitoring sensor is further configured to sense a displacement of said monitoring sensor and to transmit the sensed displacement information of said detection sensor wirelessly, when the communicator is further configured to receive the transmitted displacement information of said detection sensor wirelessly from said detection sensor.

12. The monitoring system according to claim 11, wherein the processor is further configured to determine a thickness of a layer of the concrete on the grounds of the received displacement information of said detection sensor.

13. The monitoring system according to claim 1, wherein the communicator and the processor belong to a reader, or the communicator belongs to the reader and the processor belongs to an analysis computer.

14. A monitoring method for monitoring a strength development of sprayed concrete by the monitoring system according to claim 1, comprising at least following steps of:
spraying, by the sprayer, the concrete on the target surface,
sensing, by the each monitoring sensor, the at least one strength development quantity from the sprayed concrete,
receiving, by the communicator, the at least one sensed strength development quantity-related information wirelessly from the at least one monitoring sensor, and
processing, by the processor, the at least one received strength development quantity-related information to represent the development of the strength of the concrete,
wherein the at least one monitoring sensor is sprayed, by the sprayer, on the target surface after the sprayer has been provided with the at least one monitoring sensor.
